(19) United States
(12) Patent Application Publication
(10) Pub. No.: US 2021/0385391 A1
(43) Pub. Date: Dec. 9, 2021

(54) MEDICAL VIDEO SWITCHER

(71) Applicant: Medi Plus Inc., Tokyo (JP)

(72) Inventors: Naoya Sugano, Tokyo (JP); Minsu Kwon, Tokyo (JP)

(73) Assignee: Medi Plus Inc., Tokyo (JP)

(21) Appl. No.: 17/266,022

(22) PCT Filed: Aug. 2, 2019
(86) PCT No.: PCT/JP2019/030481

§ 371 Date: Feb. 4, 2021

(30) Foreign Application Priority Data
Sep. 6, 2018 (JP) 2018-167233

(51) Int. Cl. H04N 5/268; G06F 3/0482; G06F 3/0484; H04N 7/18; A61B 90/00

(52) U.S. Cl. CPC H04N 5/268; G06F 3/0482; G06F 2203/04803; H04N 7/18; A61B 90/37; G06F 3/0484

(57) ABSTRACT

Provided is a medical video switcher more easily operable by the user, wherein a medical video switcher 100 designates one or a plurality of input frames and one or a plurality of output frames in response to a pointing operation made on a first operation screen on which a plurality of input frames that individually correspond to a plurality of input channels and a plurality of output frames that individually correspond to a plurality of output channels are displayed; correlates one input frame to any of the output frames, in response to a pointing operation made on a second operation screen on which the input frame and the output frame designated in response to a pointing operation made on the first operation screen are displayed; and connects the input channel that corresponds to the correlated input frame and the output channel that corresponds to the output frame.

### TECHNICAL FIELD

The present invention relates to a medical video switcher.

### BACKGROUND ART

Recent medical surgery has been widely practiced referring to video of surgical field and measured results of vital signs which are displayed on a plurality of monitors installed in an operating room, or recorded on a recorder, needing proper switching among videos to be output on the individual devices (monitors, recorder, etc.) during surgery.

As a prior art document that discloses a device that embodies the aforementioned switching among videos and so forth, exemplified is Patent Literature 1 below.

Patent Literature 1 discloses a medical system that automatically determines whether the monitors connected to a video switcher are 3D-compatible or not, and automatically selects a monitor compatible to the dimension of input image.

### CITATION LIST

### Patent Literature

### [Patent Literature 1] JP-A-2016-36592

### SUMMARY OF THE INVENTION

### Technical Problem

A medical video switcher (video switcher) contained in the medical system disclosed in Patent Literature 1 can highlight an operation button of an output terminal which is connected to a monitor capable of displaying video signal received through an input terminal, upon operation made on a button of the input terminal on a touch panel; or can hide an operation button of an output terminal connected to a monitor incapable of displaying the video signal received through the input terminal.

The medical video switcher of Patent Literature 1 will, however, have an increased number of buttons of the input terminals displayed on the touch panel, as the number of input terminals increases. Some of the buttons of the input terminals may be unused for switching of video and so forth during surgery. This would consequently induce human errors such as misoperation or misjudgment.

As the medical devices diversify, the number of medical devices (the number of input systems of the medical video switcher) used for every surgery tends to increase, and also the numbers of monitors and recorders installed in an operating room (the number of output systems of the medical video switcher) tend to increase. Thus the aforementioned human error is increasingly anticipated.

The present invention was arrived at in consideration of the aforementioned problem, and is to provide a medical video switcher more easily operable by the user.

### Solution to Problem

According to the present invention, there is provided a medical video switcher receiving a medical video through any of a plurality of input channels, and outputting the medical video through any of a plurality of output channels which is connected to the input channel,

the medical video switcher includes:
a display unit capable of displaying at least a first operation screen and a second operation screen; and
an operation input unit capable of accepting pointing operation made on the first operation screen and the second operation screen,

the first operation screen being provided for display of a plurality of input frames individually correlated to the plurality of input channels, and a plurality of output frames individually correlated to the plurality of output channels, and capable of accepting designation of one or a plurality of the input frames and one or a plurality of the output frames, from among the input frames and the output frames displayed on the first operation screen, in response to the pointing operation made on the first operation screen,

the second operation screen being provided for display of the input frames and the output frames designated in response to the pointing operation made on the first operation screen, and capable of accepting correlation of one input frame from among the input frames displayed on the second operation screen, with any of the output frames displayed on the second operation screen, in response to the pointing operation made on the second operation screen,

and,

the medical video switcher being devised to connect the input channel that corresponds to the input frame with the output channel that corresponds to the output frame, where the input frame and the output frame have been correlated in response to the pointing operation made on the second operation screen.

Since the present invention has the first operation screen on which the channels for use are determined and the second operation screen on which the video and so forth are switched, which are divided from each other, so that the channels not for use can be thinned out, before a phase (for example, during surgery) where switching of video and so forth is actually required. This can consequently simplify the operation regarding switching of video and so forth in such phase, and can therefore suppress human errors.

### Advantageous Effects of Invention

The present invention can provide a medical video switcher more easily operable by the user.

### BRIEF DESCRIPTION OF DRAWINGS

FIG. 1 is a configuration diagram illustrating a configuration of a medical video system of this embodiment.

FIG. 2 is a configuration diagram illustrating a configuration of a medical video switcher of this embodiment.

FIG. 3 is a drawing explaining a first operation screen displayed on a touch panel device.

FIG. 4 is a schematic drawing explaining a method of operation made on the first operation screen.

FIG. 5 is a schematic drawing explaining a method of operation made on the first operation screen.

FIG. 6 is a schematic drawing explaining a method of operation made on the first operation screen.

FIG. 7 is a schematic drawing explaining a method of operation made on the first operation screen.

FIG. 8 is a schematic drawing explaining a method of operation made on the first operation screen.

FIG. 9 is a drawing explaining a display mode of a second operation screen displayed on the touch panel device.

FIG. 10 is a schematic drawing explaining a method of operation made on the second operation screen.

FIG. 11 is a schematic drawing explaining a method of operation made on the second operation screen.

FIG. 12 is a schematic drawing explaining a method of operation made on the second operation screen.

FIG. 13 is a drawing illustrating another display mode of the second operation screen displayed on the touch panel device.

### DESCRIPTION OF EMBODIMENTS

Embodiments of the present invention will be explained below referring to the attached drawings. Note that all similar constituents in all drawings will be given same reference signs, so as to suitably avoid redundant explanation.

### <Constituents Contained in Medical Video System 1000>

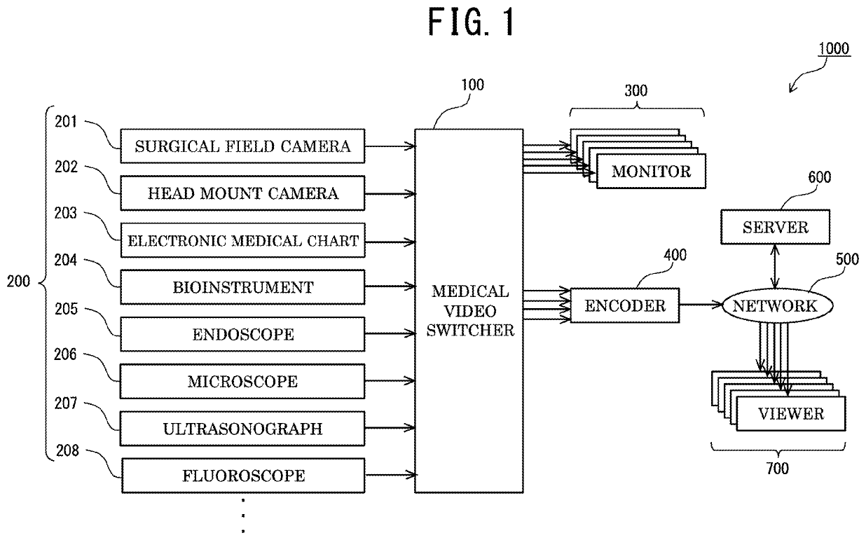

First, the individual constituents contained in a medical video system 1000 will be explained referring to FIG. 1. FIG. 1 is a configuration diagram illustrating a configuration of the medical video system 1000 of this embodiment

Arrows illustrated in FIG. 1 represent origins and destinations of video transferred among the individual constituents. Transfer of information and data other than video does not always necessarily coincide with indication of each arrow.

The medical video system 1000 has a medical video switcher 100, a medical device group 200 connected to input channels of the medical video switcher 100, and a monitor group 300 and an encoder 400 individually connected to output channels of the medical video switcher 100. The medical video system 1000 also has a server 600 that downloads and then stores video supplied from an encoder 400 through a network 500, and a viewer group 700 that requests video to the server 600 through the network 500, acquires the requested video from the server 600 through the network 500, and then displays (plays) the video.

The medical video switcher 100 is a device that receives a medical video through any of a plurality of input channels, and outputs the medical video through any of a plurality of output channels which is connected to the input channel.

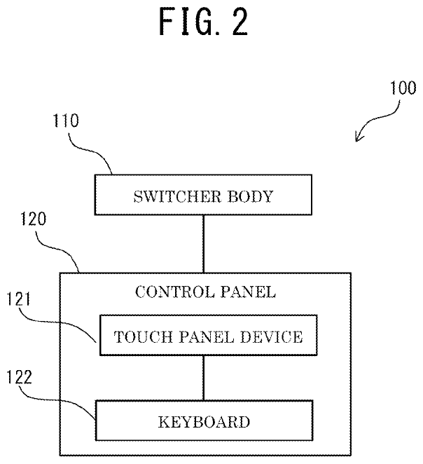

FIG. 2 is a configuration diagram illustrating a configuration of the medical video switcher 100.

Constituents of the medical video switcher 100 are roughly classified into a switcher body 110 and a control panel 120.

The switcher body 110 has a plurality of input terminals provided for every input channel, and a plurality of output terminals provided for every output channel.

In this embodiment, the switcher body 110 has twenty input terminals and twenty output terminals. That is, the switcher body 110 can receive video through up to twenty input channels, and can output video through up to twenty output channels.

Note that the numbers of the input terminals and the output terminal are not limited to the aforementioned number (twenty), but may suitably be modified.

The control panel 120 is a constituent that accepts an operation regarding the switcher body 110, wherein such operation includes at least designation of the input channels and the output channels to be connected with each other (switching of input sources and output sources). The control panel 120 has a touch panel device 121 and a keyboard 122.

The touch panel device 121 is a device that integrally includes a display device capable of displaying thereon an operation screen on which the switcher body 110 is operated, and a position entry device capable of accepting a pointing operation (touch operation) made on the operation screen. Since the operation screen includes at least a first operation screen and a second operation screen described later, so that the touch panel device 121 is understood to be a structure that embodies a display unit and an operation input unit in the present invention.

The keyboard 122 is a peripheral device connected to the touch panel device 121, and is used for a part of operation (in particular, operation in need of text entry) directed to the switcher body 110.

Note that this embodiment, although having exemplified the touch panel device 121 capable of embodying the display unit and the operation input unit as a single integrated device, may alternatively employ a configuration in which the display unit and the operation input unit are embodied as a plurality of devices (for example, a configuration having the display device and a pointing device provided separately).

Also note that this embodiment, although having exemplified a configuration that includes the keyboard 122 as hardware separate from the touch panel device 121, may alternatively employ a screen keyboard (software keyboard) displayed on the touch panel device 121, in place of or in combination with the keyboard 122.

The medical device group 200 in this embodiment is specifically exemplified by a surgical field camera 201, a head mount camera 202, an electronic medical chart device 203, a bioinstrument 204, an endoscope 205, a microscope 206, an ultrasonograph 207, and a fluoroscope 208.

The surgical field camera 201 is a camera which shoots a surgical field at a position confronted to the surgical field, and then outputs the recorded video to the medical video switcher 100.

The head mount camera 202 is a camera set on the head of an operator (surgeon), and outputs the recorded video to the medical video switcher 100.

The electronic medical chart device 203 is a computer device which stores medical chart (medical record) of a subject (patient), and outputs the stored medical chart to the medical video switcher 100. In this embodiment, not only a single medical chart, but also a plurality of medical charts may be output from the electronic medical chart device 203. The electronic medical chart device 203, when outputting a plurality of medical charts, can output the medical charts while dividing them so as to be directed to every input channel of the medical video switcher 100.

The bioinstrument 204 is a measurement instrument which measures vital signs (blood pressure, pulse rate, electrocardiogram, breathing rate, body temperature, etc.) of the subject, and output the measured vital signs to the medical video switcher 100.

The endoscope 205 is a medical device that has a lens of camera, a forceps hole and so forth mounted on the front end of a tube to be inserted into the body of subject, and outputs video captured by using the camera to the medical video switcher 100.

The microscope 206 is a medical device that enlarges a very small part (nerve, blood vessel and so forth of the subject) up to a size visible to the naked eyes, and displays and shoots it, and is capable of outputting the enlarged captured video to the medical video switcher 100.

The ultrasonograph 207 is a medical device that applies ultrasonic wave to a target (thoracic, abdomen and so forth of the subject) and visualizes the reflection, and is also called “echo”. The ultrasonograph 207 can output the video generated from the reflected ultrasonic wave to the medical video switcher 100.

The fluoroscope 208 is a medical device that irradiates X-ray to a target (the subject), produces X-ray image on the basis of transmitted X-ray, and can output the produced X-ray image to the medical video switcher 100.

The medical device group 200 exemplified here is merely a specific example, where embodiments of the present invention may lack a part of these components, or may contain any other medical device having not been mentioned above.

Note that video data regarding video and so forth, produced in the present embodiment by the medical device group 200 (including medical record and measured results of vital signs), and video that is displayed on a device having such video data entered therein (monitor group 300 and viewer group 700 described later) and so forth, are considered to fall in the category of “medical-related video”, and will be thus described below.

The monitor group 300 includes display devices that receive video data output from the medical device group 200, and displays the received video data.

Note that the number of display devices that configure the monitor group 300 is not specifically limited, so that even a single display device is also acceptable.

The encoder 400 receives the video data which is output from the medical device group 200 and allowed to pass through the medical video switcher 100, and put the received video data into encoding (coding process), so as to covert the format suitable for transfer to, and storage in the server 600.

The encoder 400 in this embodiment has four input systems, can receive the video data through each of four output channels of the medical video switcher 100, can encode the individual video data, and can store them in the server 600.

Note that the number of the input system of the encoder 400 is not specifically limited, and may be smaller or larger than four.

The network 500 may be configured by various computer networks such as the Internet, local area network (LAN) and so forth, or combinations of them. Connection for communication among the individual components contained in the network may be any of wired communication, wireless communication, or combination of them.

The viewer group 700 is computer equipment having the right to access the server 600, and can request display (playback) of video data stored in the server 600, depending on the access right. The server 600 distributes the requested video through the network 500, in response to the request for display issued by the viewer group 700.

Note that the mode of playback of video by the viewer group 700 may be either download playback or streaming playback.

Note although the explanation below presupposes that the display device equivalent to the monitor group 300 is installed in an operation room, and the computer equipment equivalent to the viewer group 700 is installed outside of the operation room, embodiments of the present invention is not limited thereto.

At least a part of the display device equivalent to the monitor group 300 may be installed outside of the operating room, or at least a part of the computer equipment equivalent to the viewer group 700 may be installed in the operation room.

### <Operation Screens Displayed on Touch Panel Device 121>

The operation screens displayed on the touch panel device 121 contain at least a first operation screen and a second operation screen.

The first operation screen is an operation screen that accepts, for example, an operation of designating the input channel and the output channel assigned to videos to be displayed on the second operation screen.

The second operation screen is an operation screen that displays thereon videos assigned to the input channel and output channel designated on the first operation screen, and accepts for example an operation for switching the input channel and the output channel to be connected.

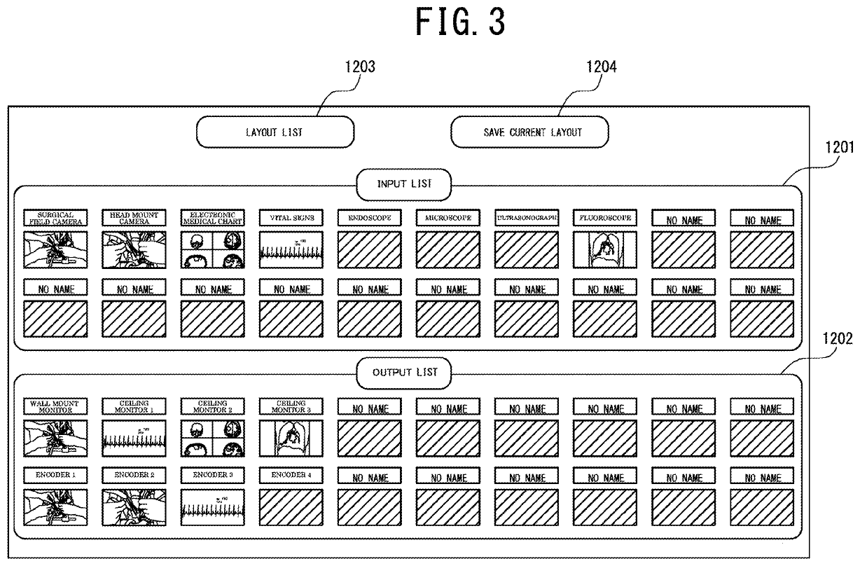

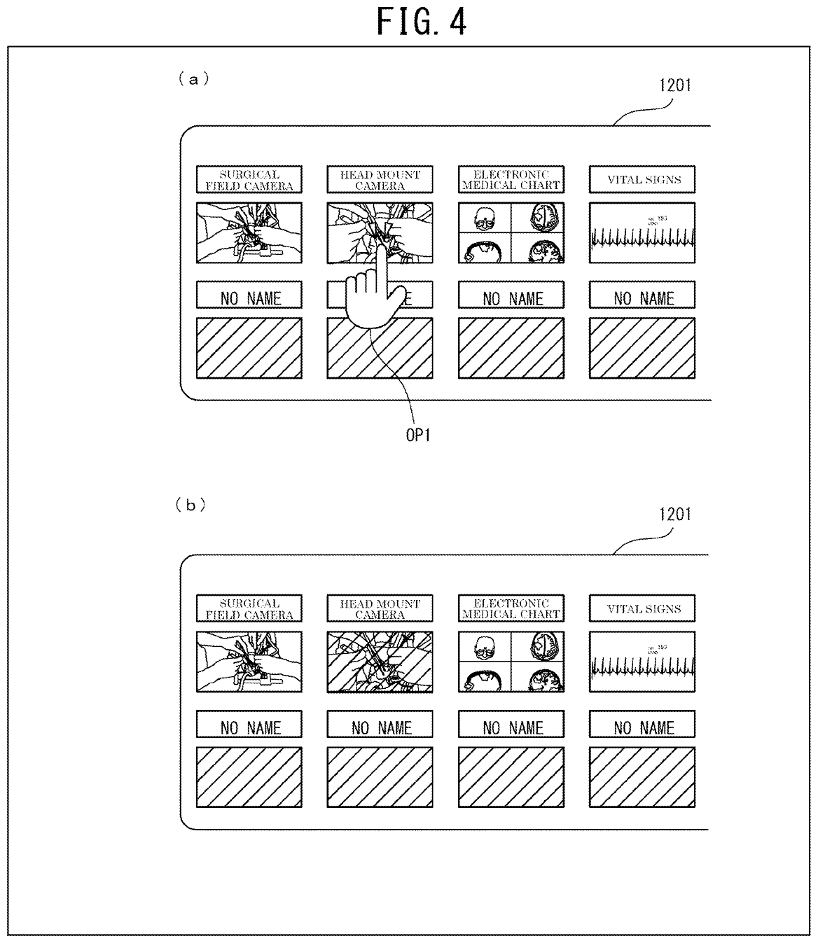

FIG. 3 is a drawing illustrating the first operation screen displayed on the touch panel device 121. FIGS. 4 to 8 are schematic drawings illustrating methods of operations made on the first operation screen.

On the first operation screen, there are displayed an input list 1201 that displays twenty display frames that individually correspond to each of twenty input channels (referred to as “input frames”, hereinafter), and an output list 1202 that displays twenty display frames that individually correspond to each of twenty output channels (referred to as “output frames”, hereinafter).

The user can give a freely selectable name to each of the input frames displayed in the input list 1201 and each of the output frames displayed in the output list 1202, wherein the thus assigned names are displayed on the top sides of the individual input frames and the individual output frames. Note, when the names are assigned, text entry by the user is preferably accepted on the keyboard 122.

Positions of the individual input frames and the individual output frames follow a physical layout of the input terminals and the output terminals provided to the switcher body 110.

For the convenience of the explanation below, the input frames arrayed in the upper row of the input list 1201 are named input frames A1 to A10 from left to right, for the purpose of denoting them in a discriminating manner. The input frames arrayed in the lower row of the input list 1201 are named input frames A11 to A20 from left to right, for the purpose of denoting them in a discriminating manner. The output frames arrayed in the upper row of the output list 1202 are named output frames B1 to B10 from left to right, for the purpose of denoting them in a discriminating manner. The output frames arrayed in the lower row of the output list 1202 are named output frames B11 to B20 from left to right, for the purpose of denoting them in a discriminating manner.

In FIG. 3, hatched input frames (more specifically, input frames A5 to A7 and input frames A9 to A20) and hatched output frames (more specifically, output frames B5 to B10 and output frame B14 to B20) are the frames not designated (or designation canceled) as the input frames and output frames to be displayed on the second operation screen, and the other input frames and output frames are designated as the input frames and the output frames to be displayed on the second operation screen.

The input frame A1 which corresponds to an input terminal connected to the surgical field camera 201 is named “surgical field camera”, and displays a thumbnail image of video (live video) currently supplied from the surgical field camera 201.

The input frame A2 which corresponds to an input terminal connected to the head mount camera 202 is named “head mount camera”, and displays a thumbnail image of video (live video) currently supplied from the head mount camera 202.

The input frame A3 which corresponds to an input terminal connected to the electronic medical chart device 203 is named “electronic medical chart device”, and displays a thumbnail image of video (CT image as a medical chart of subject) currently supplied from the electronic medical chart device 203.

The input frame A4 which corresponds to an input terminal connected to the bioinstrument 204 is named “vital signs”, and displays a thumbnail image of video (measurement result of vital signs) currently supplied from the bioinstrument 204.

The input frame A8 which corresponds to an input terminal connected to the fluoroscope 208 is named “fluoroscope”, and displays a thumbnail image of video (X-ray image of subject) currently supplied from the fluoroscope 208.

The input frame A6 which corresponds to an input terminal connected to the endoscope 205 is named “endoscope”, but the thumbnail image thereof is not displayed since it is not designated as an input frame to be displayed on the second operation screen.

The input frame A7 which corresponds to an input terminal connected to the microscope 206 is named “microscope”, but the thumbnail image thereof is not displayed since it is not designated as an input frame to be displayed on the second operation screen.

The input frame A8 which corresponds to an input terminal connected to the ultrasonograph 207 is named “ultrasonograph”, the thumbnail image thereof is not displayed since it is not designated as an input frame to be displayed on the second operation screen.

The input frame A9 to A20 which correspond to the other input terminals have no freely selectable name assigned by the user, and are not designated as input frames to be displayed on the second operation screen (there are no medical device group 200 which corresponds to the input channels), so that the thumbnail images are not displayed.

The output frame B1 which corresponds to an output terminal connected to a wall mount monitor (not illustrated), which is one constituent of the monitor group 300, is named “wall mount monitor”. At a point in time illustrated in FIG. 3, the output frame B1 is correlated to the input frame A1, in which a thumbnail image of video (video taken by the surgical field camera 201) currently supplied to the input frame A1 is displayed.

The output frame B2 which corresponds to an output terminal connected to a ceiling monitor (not illustrated), which is one constituent of the monitor group 300, is named “ceiling monitor 1”. At a point in time illustrated in FIG. 3, the output frame B2 is correlated to the input frame A4, in which a thumbnail image of video (measurement result of vital signs) currently supplied to the input frame A4 is displayed.

The output frame B3 which corresponds to an output terminal connected to a ceiling monitor (not illustrated), which is one component of the monitor group 300 and is connected to a ceiling monitor (not illustrated) other than the aforementioned ceiling monitor, is named “ceiling monitor 2”. At a point in time illustrated in FIG. 3, the output frame B3 is correlated to the input frame A3, in which a thumbnail image of video (CT image as medical chart of subject) currently supplied to the input frame A3 is displayed.

The output frame B4 which corresponds to an output terminal connected to a ceiling monitor (not illustrated), which is one component of the monitor group 300 and is connected to a ceiling monitor (not illustrated) again other than the aforementioned ceiling monitors, is named “ceiling monitor 3”. At a point in time illustrated in FIG. 3, the output frame B4 is correlated to the input frame A8, in which a thumbnail image of video (X-ray image of subject) currently supplied to the input frame A8 is displayed.

The output frame B11 which corresponds to an output terminal connected to a first input system of the encoder 400 is named “encoder 1”. At a point in time illustrated in FIG. 3, the output frame B11 is correlated to the input frame A1, in which a thumbnail image of video (video taken by the surgical field camera 201) currently supplied to the input frame A1 is displayed.

The output frame B12 which corresponds to an output terminal connected to a second input system of the encoder 400 is named “encoder 2”, and is correlated, at a point in time illustrated in FIG. 3, to the input frame A2, in which a thumbnail image of video (video taken by the surgical head mount camera 202) currently supplied to the input frame A2 is displayed.

The output frame B13 which corresponds to an output terminal connected to a third input system of the encoder 400 is named “encoder 3”, and is correlated, at a point in time illustrated in FIG. 3, to the input frame A4, in which a thumbnail image of video (measurement result of vital signs) currently supplied to the input frame A4 is displayed.

The output frame B14 which corresponds to an output terminal connected to a fourth input system of the encoder 400 is named “encoder 4”, but the thumbnail image thereof is not displayed since it is not designated as an output frame to be displayed on the second operation screen.

The output frames B5 to B10 and B15 to B20 which correspond to the other output terminals have no freely selectable name assigned by the user, and are not designated as input frames to be displayed on the second operation screen, so that the thumbnail images are not displayed.

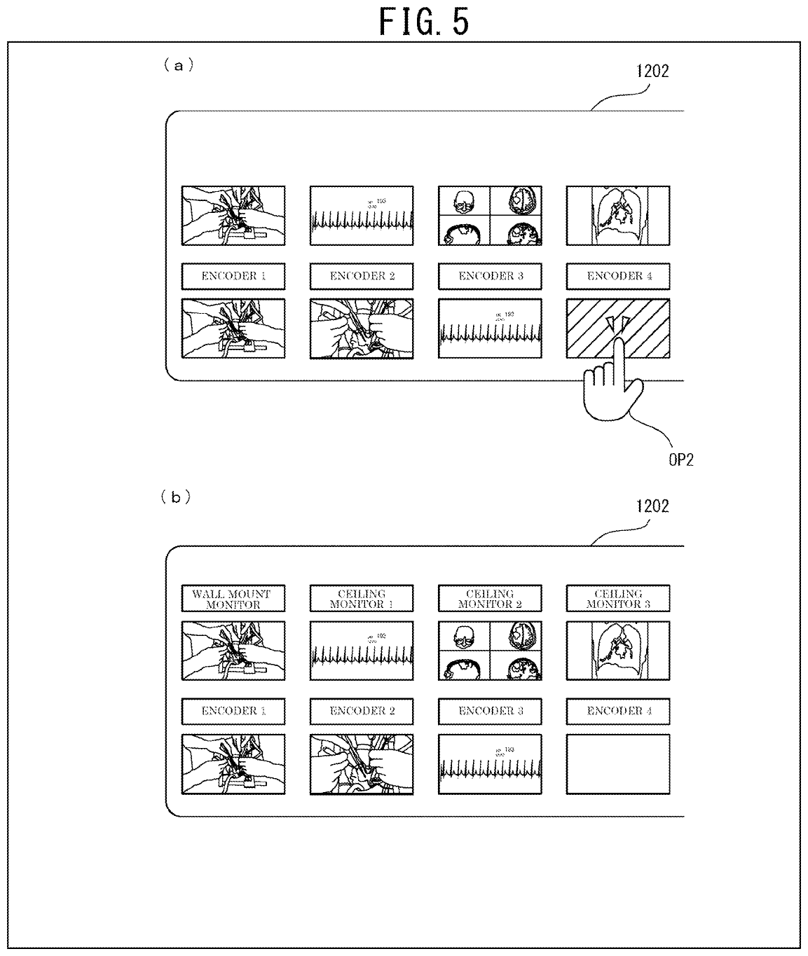

A specific example of the method of operation made on the first operation screen (method of operation for designating the input channels and the output channels regarding videos to be displayed on the second operation screen) is illustrated in FIGS. 4 and 5.

In this embodiment, the input frame or the output frame to be displayed on the second operation screen may be created, by designating one or a plurality of input frames and one or a plurality of output frames, from among the input frames and the output frames displayed on the first operation screen, in response to the pointing operation made on the first operation screen. Now, “the pointing operation made on the first operation screen” specifically means touching quickly twice (double-tap) on a display area where an input frame or an output frame to be designated is displayed. Note that finger point patterns OP1 and OP2 in FIGS. 4(a) and 5(a) merely symbolize motion of a user's hand, and do not practically appear on the touch panel device 121.

Also note that the double-tap explained here is a specific example of operation for designating an input frame or an output frame to be displayed on the second operation screen, while allowing any other pointing operation to be implemented in place of, or in combination with, the aforementioned operation to effect a similar process.

In this embodiment, double-tap made on the input frame A2 as illustrated in FIG. 4(a) will make a thumbnail image of video, being displayed on the input frame A2 (video taken by the head mount camera 202), less visible (for example, the thumbnail image will become less bright). This makes the user recognize that designation has been canceled (designation has not been made) (see FIG. 4(b)).

Alternatively, double-tap made on the output frame B14 as illustrated in FIG. 5(a) will change a mode of display on the output frame B14 (for example, the output frame B14 will become brighter). This makes the user recognize that designation has been made (see FIG. 5(b)).

Note that although the designated input frame (or output frame), and the undesignated input frame (or output frame) are preferably displayed in discriminable modes, the modes of display are not limited to the aforementioned embodiment. For example, presence or absence of designation may be displayed separately from the input frame and the output frame; or presence of absence of designation may be represented by a part of the input frame or the output frame (color of the frame contour, etc.).

As has explained referring to FIGS. 4 and 5, the input frame or the output frame to be displayed on the second operation screen may be created, by designating one or a plurality of input frames and one or a plurality of output frames, from among the input frames and the output frames displayed on the first operation screen, irrespective of presence or absence of display of the thumbnail image (input of video and so forth) in the input frames and the output frames on the first operation screen. Therefore, even if there were, for example, the input frame to which video is not received from the medical device group 200 over a duration where the first operation screen is displayed, and the output frame correlated to the input frame, these frames may be displayed on the second operation screen.

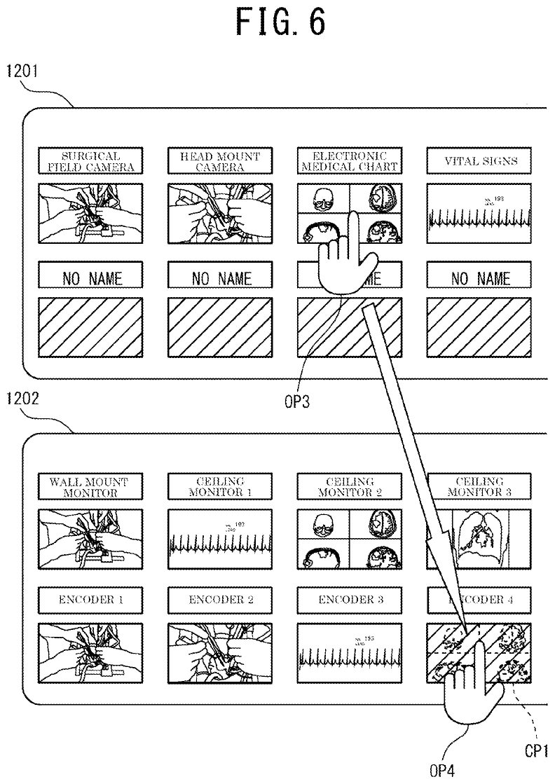

A specific example of the method of operation made on the first operation screen (method of operation for correlating the input frame displayed on the first operation screen, with the output frame displayed on the first operation screen) is illustrated in FIGS. 6 to 8.

Finger point patterns OP3 and OP4 in FIG. 6 merely symbolize motion of a user's hand in a drag-and-release operation, and do not practically appear on the touch panel device 121. Now, the drag-and-release operation is specifically a method that includes accepting, on the first operation screen, a pointing operation directed to one input frame A3; then shifting the position of the pointing operation from the input frame A3 to one output frame B14 while sustaining the pointing operation; and terminating the pointing operation on the output frame B14. Upon acceptance of such operation method, the medical video switcher 100 correlates the input frame A3 to the output frame B14. On the first operation screen in this case, when the pointing operation directed to the input frame is sustained, a copied image CP1 of the thumbnail image displayed in the input frame moves so as to follow shifting of the pointing operation, and the copied image CP1 of the thumbnail image disappears upon termination of the pointing operation,

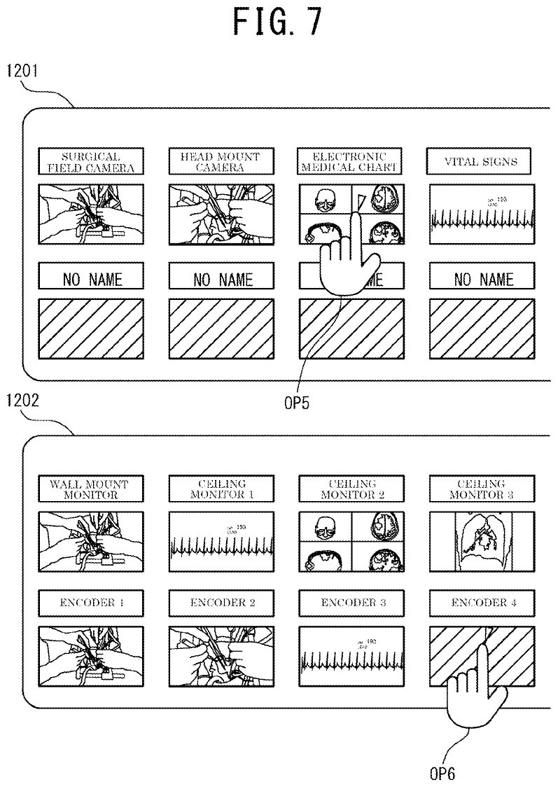

Meanwhile, finger point patterns OP5 and OP6 illustrated in FIG. 7 symbolize motion of a user's hand in a touch operation made only once (one-tap operation) on each of the input frame A3 and the output frame B14, and do not practically appear on the touch panel device 121. Now the “one-tap operation” is specifically a method that includes accepting, on the first operation screen, a pointing operation directed to one input frame A3; and then newly accepting a pointing operation directed to one output frame B14. Also upon acceptance of such operation method, the medical video switcher 100 correlates the input frame A3 to the output frame B14. Now, this case, when compared with the case illustrated in FIG. 6 (where drag-and-release operation is accepted), is different in that the copied image (equivalent to the copied image CP1) of the thumbnail image displayed in the input frame A3 is not displayed, over the duration from acceptance of the pointing operation directed to one input frame A3 up to acceptance of new pointing operation directed to one output frame B14.

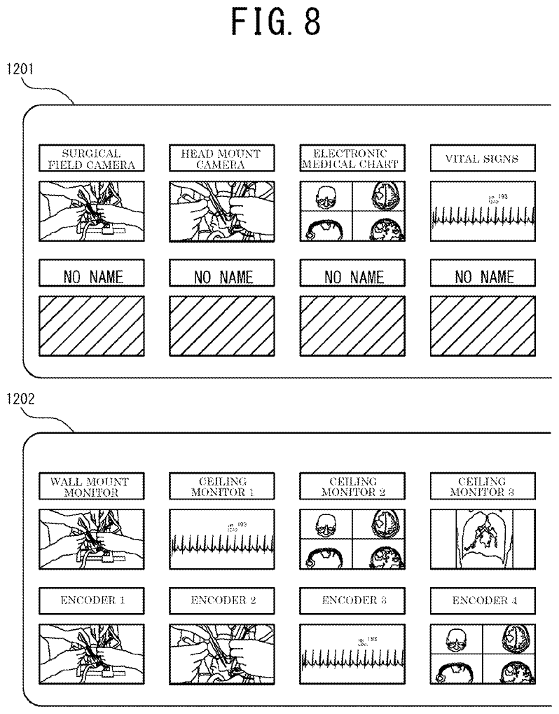

In either of the aforementioned cases, the input frame A3 and the output frame B14 are correlated, and the output frame B14 will have displayed therein a thumbnail image of video currently supplied to the input frame A3 (CT image of subject) as a consequence (see FIG. 8).

The “drag-and-release operation” and the “one-tap operation” made on the first operation screen are intended to display the first operation screen in a preparation phase of surgery (before display of the second operation screen), and to correlate the output frame and the input frame at the beginning of surgery (upon display of the second operation screen).

Note that the aforementioned “drag-and-release operation” and the “one-tap operation” are merely specific examples of the operation for correlating the input frame displayed on the first operation screen, to the output frame displayed on the first operation screen, while allowing any other pointing operation to be implemented in place of, or in combination with, the aforementioned operation to effect a similar process.

On the first operation screen, there are provided a layout list display button 1203 and a layout save button 1204, besides the aforementioned input list 1201 and the output list 1202.

The layout save button 1204 is pointed when saving the current layout on the first operation screen (more specifically, correlation between the input frame and the output frame on the first operation screen, names assigned to the input frame and the output frame on the first operation screen, and presence or absence of designation regarding the input frame and the output frame). The layout to be saved may be named. Different layouts may be saved, while classified by operative method, surgeon and so forth.

The layout list display button 1203 is pointed when displaying a list of layout (not illustrated) of the first operation screen ever saved. Upon designation of any layout displayed in the list, the first operation screen is changed to display the new layout thus designated.

As explained above, the first operation screen can accept the pointing operation (pointing operation illustrated in FIGS. 6 and 7) that correlates the input frame being displayed on the first operation screen, to the output frame being displayed on the first operation screen.

The first operation screen can also accept the pointing operation (pointing operation illustrated in FIGS. 4 and 5) that designates the input frame and the output frame to be displayed on the second operation screen, from among the input frames and the output frames being displayed on the first operation screen.

Again, the first operation screen can also accepts the pointing operation (pointing operation directed to the layout save button 1204) that saves, at least, correlation between the input frame and the output frame in response to the first pointing operation, and designation regarding the input frame and the output frame in response to the second pointing operation.

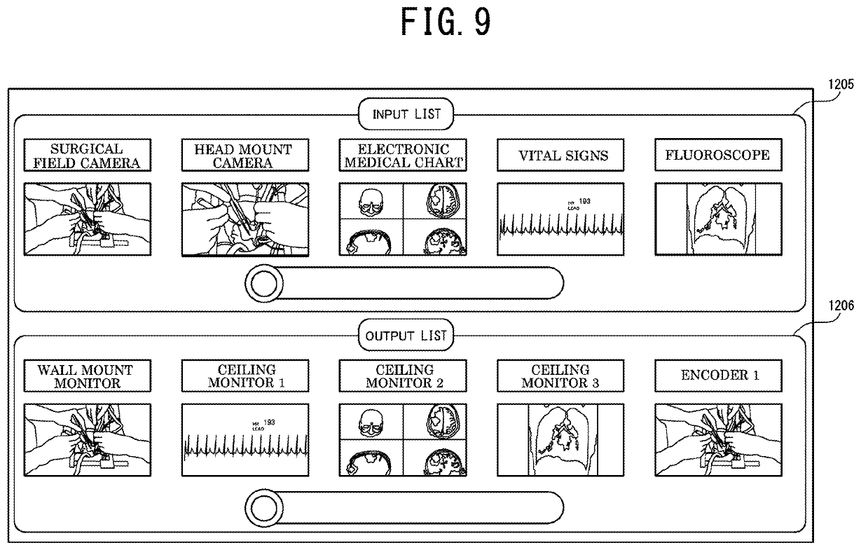

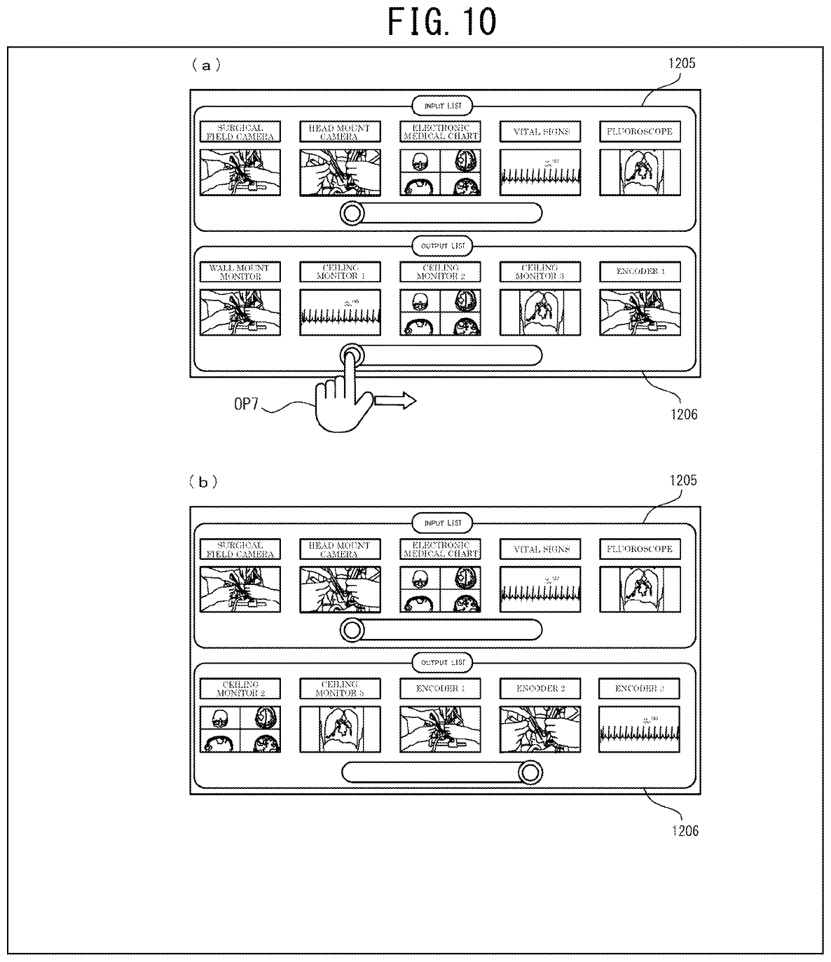

FIG. 9 is a drawing illustrating the second operation screen displayed on the touch panel device 121. FIGS. 10 to 12 are schematic drawings explaining a method of operation to be made on the second operation screen.

The second operation screen is provided for display of the input frames and the output frames designated in response to the pointing operation made on the first operation screen, wherein the individual input frames are displayed in an input list 1205, and the individual output frames are displayed in an output list 1206.

For convenience of explanation below, the input frames arrayed in the input list 1205 are named input frames C1 to C5 from left to right, for the purpose of denoting them in a discriminating manner. The output frames arrayed in the output list 1206 are named output frames D1 to D5 from left to right, for the purpose of denoting them in a discriminating manner.

The second operation screen illustrated in FIG. 9 is on the premise of the arrangement explained referring to FIG. 3.

More specifically, the input list 1205 contains the input frame C1 displayed upon designation directed to the input frame A1, the input frame C2 displayed upon designation directed to the input frame A2, the input frame C3 displayed upon designation directed to the input frame A5, the input frame C4 displayed upon designation directed to the input frame A11, and the input frame C5 displayed upon designation directed to the input frame A12.

Meanwhile, the output list 1206 contains the output frame D1 displayed upon designation directed to the output frame B1, the output frame D2 displayed upon designation directed to the output frame B2, the output frame D4 displayed upon designation directed to the output frame B3, the output frame D4 displayed upon designation directed to the output frame B4, and the output frame D5 displayed upon designation directed to the output frame B11.

As illustrated in FIG. 9, the input frames displayed in the input list 1205 and the output frames displayed in the output list 1206 are given the names having been assigned to the individually correlated input frames displayed in the input list 1201, and the names having been assigned to the individually correlated output frames displayed in the output list 1202.

As is clear from comparison between FIG. 3 and FIG. 9, the input frames and the output frames on the second operation screen display are the input frames and the output frames designated on the first operation screen, which are justified to the left. In other words, the layout of the input frames and the output frames displayed on the second operation screen is made compact, as compared with the layout of the input frames and the output frames designated on the first operation screen.

Note that the layout of the input frames and the output frames displayed on the second operation screen may be changed not only in a left-justified manner as described in the above embodiment, but may also in any manner so long as it may recognizable by the user more easily than the layout of the input frames and the output frames displayed in the first operation screen.

As illustrated in FIG. 9, only five each of the input frames and the output frames are displayed on the second operation screen, which is less than the number of the input frames and the output frames (twenty each) displayed on the first operation screen. As a consequence, the input frames and the output frames displayed on the second operation screen can be enlarged as compared with the input frames and the output frames displayed on the first operation screen. Hence, the input frames and the output frames displayed on the second operation screen can be more recognizable by the user, as compared with the input frames and the output frames displayed on the first operation screen.

As illustrated in FIG. 9, there are slide bars arranged in a lower part of the input list 1205 and in a lower part of the output list 1206. By laterally sliding a button of each slide bar, the input frames or the output frames having been hidden outside of the display area of the second operation screen will now be brought into the display area of the second operation screen.

FIG. 10 illustrates an exemplary operation made on the slide bar. Note that a finger point pattern OP7 illustrated in FIG. 10(a) symbolizes a rightward sliding motion of a user's hand, and does not practically appear on the touch panel device 121.

In this embodiment, upon rightward sliding of the slide bar in the output list 1206 according to the operation method illustrated in FIG. 10(a), it now becomes possible to bring the output frames having been hidden outside of the display area, more specifically, an output frame B12 displayed upon designation directed to the output frame D6, and an output frame B13 displayed upon designation directed to the output frame D7, into the display area (see FIG. 10(b)).

Operation on the slide bar explained in this embodiment is one exemplary operation for displaying the input frames or the output frames, having been hidden outside of the display area, wherein swipe operation on the input list 1205 or the output list 1206 (operation of laterally rubbing the screen of the touch panel device 121) may be implemented in place of, or in combination with, the aforementioned operation to effect a similar process.

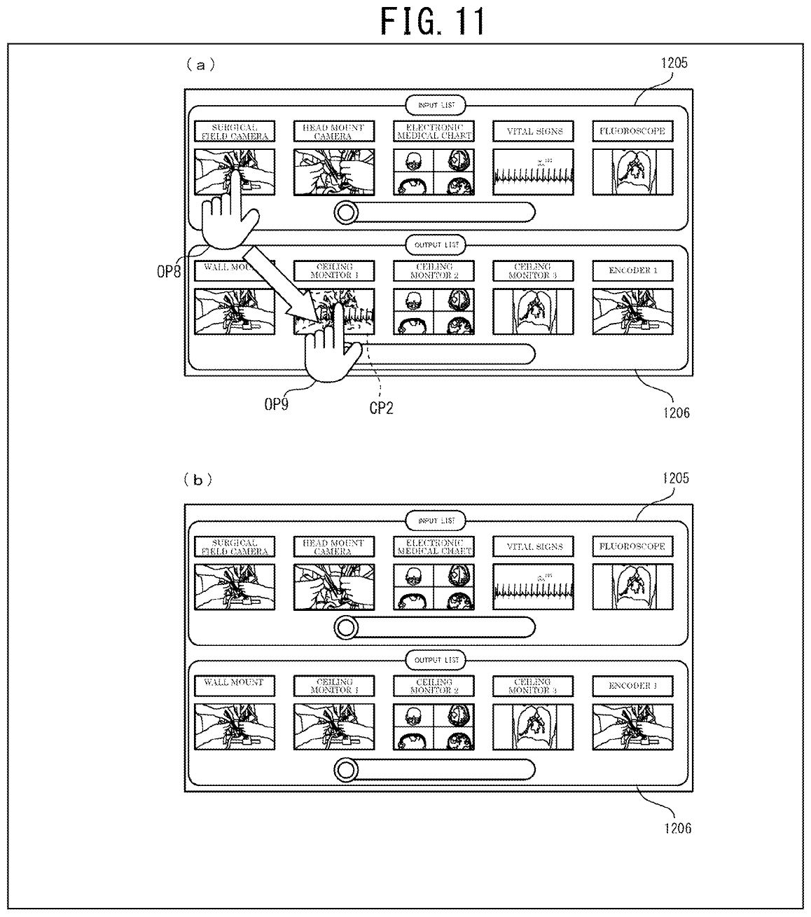

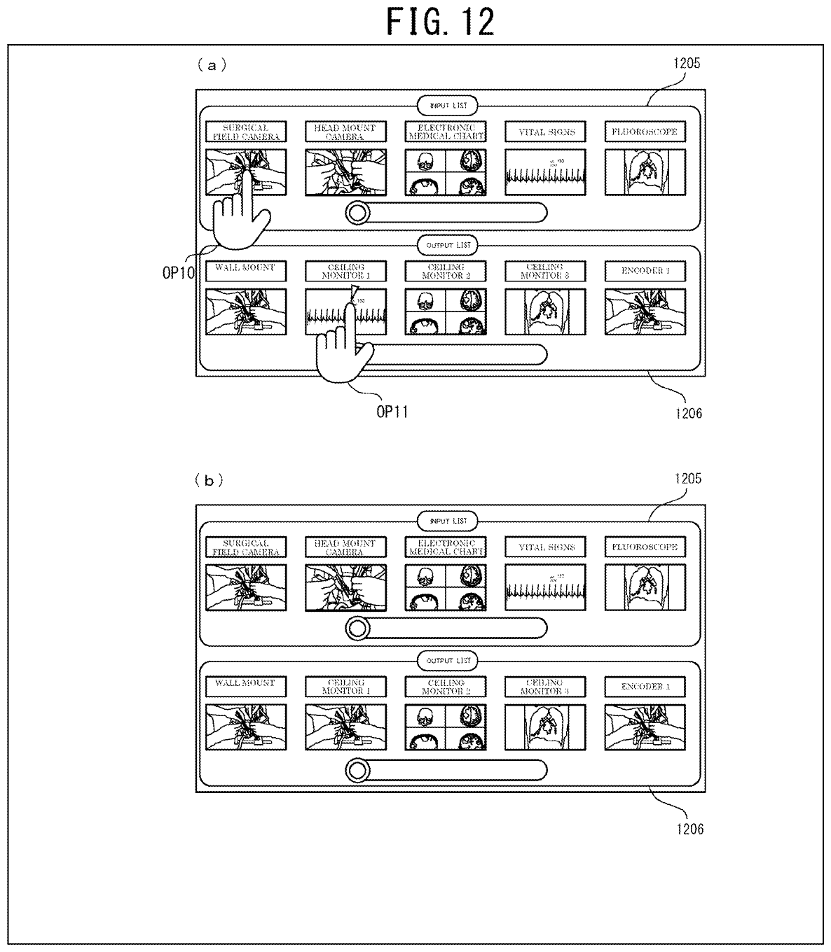

The medical video switcher 100 can correlate one input frame from among the input frames displayed on the second operation screen, with any of the output frames displayed on the second operation screen, in response to the pointing operation made on the second operation screen, and can connect the input channel that corresponds to the input frame with the output channel that corresponds to the output frame, where the input frame and the output frame have been correlated in response to the pointing operation made on the second operation screen. Now, the “pointing operation made on the second operation screen” specifically includes two patterns illustrated in FIGS. 11 and 12.

FIG. 11 illustrates a specific example of the aforementioned “pointing operation made on the second operation screen”. Note that a finger point patterns OP8 and OP9 illustrated in FIG. 11(a) symbolize a user's hand in the drag-and-release operation, and do not practically appear on the touch panel device 121.

The “drag-and-release operation” is specifically a method that includes accepting, on the second operation screen, a pointing operation directed to one input frame C1; then shifting the position of the pointing operation from the input frame C1 to one output frame D2 while sustaining the pointing operation; and terminating the pointing operation on the output frame D2. Upon acceptance of such operation method (referred to as “first case”, hereinafter), the medical video switcher 100 correlates the input frame C1 to the output frame D2.

In more detail, in the aforementioned first case, when the pointing operation directed to the input frame is sustained on the second operation screen, a copied image CP2 of the thumbnail image displayed on the input frame moves so as to follow shifting of the pointing operation, and upon termination of the pointing operation, the copied image CP2 of the thumbnail image disappears.

FIG. 12 illustrate another specific example of the aforementioned “pointing operation made on the second operation screen”. Note that finger point patterns OP10 and OP11 illustrated in FIG. 12(a) symbolize motion of a user's hand in a touch operation made only once (one-tap operation) on each of the input frame C1 and the output frame D2, and do not practically appear on the touch panel device 121.

Now the “one-tap operation” is specifically a method that includes accepting, on the second operation screen, a pointing operation directed to one input frame C1; and then newly accepting a pointing operation directed to one output frame D2. Also upon acceptance of such operation method (referred to as “second case”, hereinafter), the medical video switcher 100 correlates the input frame C1 to the output frame D2.

Now, the second case, when compared with the first case (where drag-and-release operation is accepted), is different in that the copied image (equivalent to the copied image CP2) of the thumbnail image displayed in the input frame C1 is not displayed, over the duration from acceptance of the pointing operation directed to one input frame C1 up to acceptance of new pointing operation directed to one output frame D2.

The “drag-and-release operation” and the “one-tap operation” made on the second operation screen are intended to correlate the output frames and the input frames during surgery (upon display of the second operation). That is, it is intended to a situation where more labor is consumed by other works, and videos are more frequently switched, than in the preparation phase of surgery (phase with the first operation screen displayed), making the operation easier as a result of thinning, in the preparation phase, of the input frames and the output frames having been displayed on the first operation screen.

Note that the aforementioned “drag-and-release operation” and the “one-tap operation” are merely specific examples of the operation for correlating the input frame displayed on the second operation screen, to the output frame displayed on the second operation screen, while allowing any other pointing operation to be implemented in place of, or in combination with, the aforementioned operation to effect a similar process.

Upon acceptance of either operation illustrated in FIG. 11(a) or FIG. 12(a) (either the first case or the second case), the thumbnail image displayed in the output frame D2 is switched from the measurement result of the bioinstrument 204 to the video captured by the surgical field camera 201 as illustrated in FIGS. 11(b) and 12(b).

Since similar process for switching videos (correlating the input frame C1 and the output frame D2) may be embodied in multiple ways as described above, the users can switch videos individually according to their own tastes.

Moreover, the mode of display looks different (in terms of presence or absence of the copied image CP2) between the cases where the individual operations are accepted, and the user can visually discriminate which operation is implemented, making it possible to suppress human error such as misoperation or visual misjudgment by the user.

As illustrated in FIGS. 11(b) and 12(b), the output frame D1 and the output frame D2 will have displayed therein the thumbnail images based on the same content (video captured by the surgical field camera 201 for both frames), and therefore the videos with the same content can be output through the output channels that correspond to the output frame D1 and the output frame D2. In other words, when a plurality of output frames displayed on the second operation screen are correlated to one input frame displayed on the second operation screen, the medical video switcher 100 outputs the video received through an input channel that corresponds to the input frame, through each of the output channels that correspond to the plurality of output frames.

As explained above, since the touch panel device 121 can display the thus designed first operation screen and the second operation screen independently from each other, so that the user can switch videos received from the medical device group 200 referring to the display. The switching operation may thus be simplified, and misoperation by the user may be suppressed as a consequence.

### Modified Examples of this Invention

Having explained the present invention referring to the embodiments illustrated by using the individual drawings, the present invention is not limited to the aforementioned embodiments, instead including various modifications and improvements so long as the goal of this invention will be arrived at.

The medical video switcher of the present invention may be used not only in a case where it is employed as a constituent of the medical video system 1000 illustrated in FIG. 1, but also may be used as a constituent of other system. In other words, the medical video switcher of the present invention can establish itself as a single entity, irrespective of a configuration of peripheral equipment.

The configuration of the medical video switcher of the present invention is not limited to that illustrated in FIG. 2, and may be properly modified without spoiling the purpose of this invention. For example, the switcher body 110 and the control panel 120 may be configured integrally into a single body (single equipment).

The second operation screen of the medical video switcher of the present invention is not limited to the embodiment that contains restrictively five input frames and five output frames as illustrated in FIG. 9, wherein the number of them may be variable. In this case, the numbers of the input frames and the output frames displayed on the second operation screen are not necessarily same, instead allowing the one prevails the other.

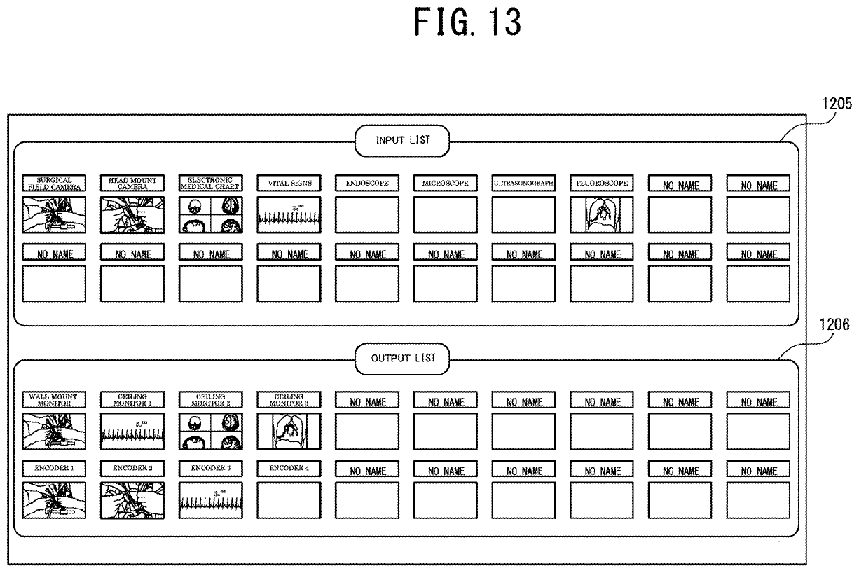

In an exemplary case where all of the input frames and the output frames displayed on the first operation screen as illustrated in FIG. 3 are designated to be displayed on the second operation screen, the number of the input frames and the output frames that can collectively be displayed on the second operation screen may be adjustable to twenty each, as illustrated in FIG. 13. On-screen size of the input frames and the output frames displayed on the second operation screen may be equivalent to the on-screen size of the number of the input frames and output frames displayed on the first operation screen. In other words, the input frames and the output frames displayed on the second operation screen, whose numbers are smaller than the numbers of the input frames and the output frames displayed on the first operation screen (for example, such as a case illustrated in FIG. 9), are preferably displayed while enlarged from these input frames and the output frames. Meanwhile, the input frames and the output frames displayed on the second operation screen, whose numbers are equal to the numbers of the input frames and the output frames displayed on the first operation screen (for example, such as a case illustrated in FIG. 13), are preferably displayed in a nearly same size as these input frames and the output frames.

Note that in a case where the numbers of the input frames and the output frames displayed on the second operation screen are made smaller than those on the first operation screen, as a result of adjustment of the numbers of the input frames and the output frames collectively displayed on the second operation screen as described above, the layout of the input frames and the output frames displayed on the second operation screen is preferably made more compact for higher visibility, as compared with the layout of the input frames and the output frames designated on the first operation screen.

This embodiment encompasses technical spirits below.

(1) A medical video switcher receiving a medical-related video through any of a plurality of input channels, and outputting the medical-related video through any of a plurality of output channels which is connected to the input channel,

the medical video switcher comprising:
a display unit capable of displaying at least a first operation screen and a second operation screen; and
an operation input unit capable of accepting pointing operation made on the first operation screen and the second operation screen,

the first operation screen being provided for display of a plurality of input frames that individually correspond to the plurality of input channels, and a plurality of output frames that individually correspond to the plurality of output channels, and capable of accepting designation of one or a plurality of the input frames and one or a plurality of the output frames, from among the input frames and the output frames displayed on the first operation screen, in response to the pointing operation made on the first operation screen,

the second operation screen being provided for display of the input frames and the output frames designated in response to the pointing operation made on the first operation screen, and capable of accepting correlation of one input frame from among the input frames displayed on the second operation screen, with any of the output frames displayed on the second operation screen, in response to the pointing operation made on the second operation screen,

and,

the medical video switcher being devised to connect the input channel that corresponds to the input frame with the output channel that corresponds to the output frame, where the input frame and the output frame having been correlated in response to the pointing operation made on the second operation screen.

(2) The medical video switcher according to (1), wherein

a layout of the input frames and the output frames displayed on the second operation screen is made compact, as compared with a layout of the input frames and the output frames designated on the first operation screen.

(3) The medical video switcher according to (1) or (2), wherein

the input frame and the output frame displayed on the second operation screen can be enlarged, as compared with the input frame and the output frame displayed on the first operation screen.

(4) The medical video switcher according to (3), wherein

the input frame and the output frame, if the numbers thereof are smaller than those of the input frame and the output frame being displayed on the first operation screen, are displayed on the second operation screen while enlarged from the input frame and the output frame being displayed on the first operation screen,

and,

the input frame and the output frame, if the numbers thereof are equal to those of the input frame and the output frame being displayed on the first operation screen, are displayed on the second operation screen in a nearly same size as the input frame and the output frame being displayed on the first operation screen.

(5) The medical video switcher according to any one of (1) to (4), wherein

in a first case where, on the second operation screen, a pointing operation directed to one input frame is accepted, the pointing operation is sustained during shift of position of the pointing operation from the input frame to one output frame, and the pointing operation is then terminated in the output frame, then the input frame and the output frame are correlated, and

also in a second case where, on the second operation screen, a pointing operation directed to one input frame is accepted, and a pointing operation directed to another output frame is newly accepted, then the input frame and the output frame are correlated.

(6) The medical video switcher according to (5), wherein

the input frame has displayed therein a thumbnail image of video being currently input,

in the first case, when the pointing operation directed to the input frame is sustained, a copied image of the thumbnail image displayed in the input frame moves so as to follow shifting of the pointing operation, and the copied image of the thumbnail image disappears upon termination of the pointing operation,

and,

in the second case, over a duration from acceptance of the pointing operation directed to one input frame pointing operation to acceptance of the pointing operation newly directed to the one output frame, the copied image of the thumbnail image displayed in the input frame is not displayed.

(7) The medical video switcher according to (6), wherein

the thumbnail image of the video being currently input is also displayed in the output frame correlated to the input frame.

(8) The medical video switcher according to any one of (1) to (7), wherein

in a case where the plurality of output frames being displayed on the second operation screen are correlated to one input frame being displayed on the second operation screen, an image received through an input channel that corresponds to the input frame is output through each output channel that correspond to the plurality of output frames.

(9) The medical video switcher according to any one of (1) to (8), wherein

the first operation screen can accept:

a first pointing operation that correlates the input frame being displayed on the first operation screen to the output frame being displayed on the first operation screen;

a second pointing operation that designates the input frame and the output frame to be displayed on the second operation screen, from among the input frames and the output frames being displayed on the first operation screen; and

third pointing operation that saves, at least, correlation between the input frame and the output frame in response to the first pointing operation, and designation regarding the input frame and the output frame in response to the second pointing operation.

This application claims priority to Japanese Patent Application No. 2018-167233 filed on Sep. 6, 2018, the entire contents of which are incorporated by reference herein.

### REFERENCE SIGNS LIST

1000: medical video system
100: medical video switcher
110: switcher body
120: control panel
121: touch panel device
1201: input list
1202: output list
1203: layout list display button
1204: layout save button
1205: input list
1206: output list
122: keyboard
200: medical device group
201: surgical field camera
202: head mount camera
203: electronic medical chart device
204: bioinstrument
205: endoscope
206: microscope
207: ultrasonograph
208: fluoroscope
300: monitor group
400: encoder
500: network
600: server
700: viewer group

1. A medical video switcher receiving a medical-related video through any of a plurality of input channels, and outputting the medical-related video through any of a plurality of output channels which is connected to the input channel,
the medical video switcher comprising:
a display unit capable of displaying at least a first operation screen and a second operation screen; and
an operation input unit capable of accepting pointing operation made on the first operation screen and the second operation screen,
the first operation screen being provided for display of a plurality of input frames that individually correspond to the plurality of input channels, and a plurality of output frames that individually correspond to the plurality of output channels, and capable of accepting designation of one or a plurality of the input frames and one or a plurality of the output frames, from among the input frames and the output frames displayed on the first operation screen, in response to the pointing operation made on the first operation screen,
the second operation screen being provided for display of the input frames and the output frames designated in response to the pointing operation made on the first operation screen, and capable of accepting correlation of one input frame from among the input frames displayed on the second operation screen, with any of the output frames displayed on the second operation screen, in response to the pointing operation made on the second operation screen,
and,
the medical video switcher being devised to connect the input channel that corresponds to the input frame with the output channel that corresponds to the output frame, where the input frame and the output frame having been correlated in response to the pointing operation made on the second operation screen.

2. The medical video switcher according to claim 1, wherein
a layout of the input frames and the output frames displayed on the second operation screen is made compact, as compared with a layout of the input frames and the output frames designated on the first operation screen.

3. The medical video switcher according to claim 1, wherein
the input frame and the output frame displayed on the second operation screen can be enlarged, as compared with the input frame and the output frame displayed on the first operation screen.

4. The medical video switcher according to claim 3, wherein
the input frame and the output frame, if the numbers thereof are smaller than those of the input frame and the output frame being displayed on the first operation screen, are displayed on the second operation screen while enlarged from the input frame and the output frame being displayed on the first operation screen,
and,
the input frame and the output frame, if the numbers thereof are equal to those of the input frame and the output frame being displayed on the first operation screen, are displayed on the second operation screen in a nearly same size as the input frame and the output frame being displayed on the first operation screen.

5. The medical video switcher according to claim 1, wherein
in a first case where, on the second operation screen, a pointing operation directed to one input frame is accepted, the pointing operation is sustained during shift of position of the pointing operation from the input frame to one output frame, and the pointing operation is then terminated in the output frame, then the input frame and the output frame are correlated, and
also in a second case where, on the second operation screen, a pointing operation directed to one input frame is accepted, and a pointing operation directed to another output frame is newly accepted, then the input frame and the output frame are correlated.

6. The medical video switcher according to claim 5, wherein
the input frame has displayed therein a thumbnail image of video being currently input,
in the first case, when the pointing operation directed to the input frame is sustained, a copied image of the thumbnail image displayed in the input frame moves so as to follow shifting of the pointing operation, and the copied image of the thumbnail image disappears upon termination of the pointing operation,
and,
in the second case, over a duration from acceptance of the pointing operation directed to one input frame pointing operation to acceptance of the pointing operation newly directed to the one output frame, the copied image of the thumbnail image displayed in the input frame is not displayed.

7. The medical video switcher according to claim 6, wherein
the thumbnail image of the video being currently input is also displayed in the output frame correlated to the input frame.

8. The medical video switcher according to claim 1, wherein
in a case where the plurality of output frames being displayed on the second operation screen are correlated to one input frame being displayed on the second operation screen, an image received through an input channel that corresponds to the input frame is output through each output channel that correspond to the plurality of output frames.

9. The medical video switcher according to claim 1, wherein
the first operation screen can accept:
a first pointing operation that correlates the input frame being displayed on the first operation screen to the output frame being displayed on the first operation screen;
a second pointing operation that designates the input frame and the output frame to be displayed on the second operation screen, from among the input frames and the output frames being displayed on the first operation screen; and
third pointing operation that saves, at least, correlation between the input frame and the output frame in response to the first pointing operation, and designation regarding the input frame and the output frame in response to the second pointing operation.
